(19) United States
(12) Patent Application Publication
(10) Pub. No.: US 2003/0199581 A1
(43) Pub. Date: Oct. 23, 2003

(54) Boswellin compositions enhanced with 3-beta-acetyl-11-keto-beta-boswellic acid ("AKBA") industrial manufacture and uses

(76) Inventors: Allen L. Seligson, Ramona, CA (US); Ronald C. Terry, San Diego, CA (US); Milos Sovak, La Jolla, CA (US); Daniel Alexander, Modrany (CZ)

Correspondence Address: FLIESLER DUBB MEYER & LOVEJOY, LLP, FOUR EMBARCADERO CENTER, SUITE 400, SAN FRANCISCO, CA, 94111, US

(21) Appl. No.: 10/387,024

(22) Filed: Mar. 12, 2003

Related U.S. Application Data
(60) Provisional application No. 60/364,299, filed on Mar. 13, 2002.

(51) Int. Cl. C07C 67/02; A61K 31/225

(52) U.S. Cl. 514/548; 560/256

(57) ABSTRACT

The biological activity of naturally occurring boswellic acids in specific plant extracts is enhanced by their peracetylation, preferably followed by silica gel treatment, or by peracetylation and mild oxidation to increase the ratio of 3-β-acetyl-11-keto-β-boswellic acid to β-boswellic acid, 3-β-acetyl-β-boswellic acid and 11-keto-β-boswellic acid. The enriched compositions compared to the commercial extracts have enhanced biological activity toward a variety of proliferative and/or inflammatory afflictions in mammalian hosts and demonstrate synergism with other phytochemicals.

### RELATED APPLICATION

This application claims priority under 35 U.S.C. §119 to U.S. Provisional Patent Application Serial No: 60/364,299, filed Mar. 13, 2002, incorporated herein fully by reference.

### BACKGROUND OF THE INVENTION

1. Field of the Invention

The invention relates to pharmacologically novel compositions derived from natural products, particularly boswellic acid complex and uses thereof. Specifically, this invention relates to natural boswellic acid complex where the ratio of the components has been altered by enrichment. More specifically, this invention relates to industrial scale production of boswellic acids and pharmaceutical uses of boswellic acids and other phytochemicals.

2. Related Art

Phytochemicals extracted from Boswellia serrata have been reported to be active in the treatment of numerous afflictions and maladies. The biological activity of the extract has been related to the components of boswellic acid complex (“boswellin”), shown to inhibit 5-lipoxygenase and leukocyte elastase. Since 5-lipoxygenase is a key enzyme in leukotriene synthesis and the leukotrienes are active agents in the inflammatory process, boswellin serves as a non-steroidal anti-inflammatory agent.

The Boswellia serrata acidic fraction comprised of pentacyclic triterpene acids has four major components: β-boswellic acid (3-β-hydroxyurs-12-en-23-oic acid) (“BA”); 3-β-acetyl-β-boswellic acid (3-β-acetoxyurs-12-en-23-oic acid) (“ABA”); 11-keto-β-boswellic acid (3-β-hydroxyurs-12-en-1-keto-23-oic acid) (“KBA”); and 3-β-acetoxy -11-keto-β-boswellic acid (3-β-acetoxyurs-12-en-11-keto-23-oic acid) (“AKBA”). In a number of in vitro and in vivo studies to be described below, altering the ratio of these specific ingredients has a profound effect on the activity of the mixtures. It is therefore of interest to devise methods that allow for the enrichment of AKBA and reduction of other boswellic acid components in the boswellin extracted from the plants, employing procedures that allow for the product to be considered GRAS (generally regarded as safe).

Boswellin, The Anti-inflammatory Phytonutrient, eds. Muhammed Majeed, Vladimir Badamaev, S. Gopinathan, R. Rajendran, and Todd Norton, Nutrascience Publishers, Inc.,121 Ethel Road West, Unit 6, Piscataway, N.J. 08854, 1996, provide a history of the gum extract from Boswellia serrata, its components and their reported biological activity. References reporting activity with 5-lipoxygenase (“5-LO”)include: Safayhi, et al., J. Pharmacol. Exp. Ther., 1992, 261, 1143-6; Safayhi, et al., Am. Soc. Pharm. Exp. Thera., 1995, 47, 1212-6; Sailer, et al., Brit. J. of Pharmacology, 1996, 117, 615-8; Ghosh and Myers, Biochem. Biophys. Res. Comm., 1997,235,418-23; and Sailer, et al., Eur. J. Biochem. 1998, 256, 364-368.

Activity of the 5-LO inhibitory action related to work up is described by Schweizer, et al., J. Nat. Prod. 2000, 63, 1058-61.

Articles that report activity of the boswellic acids toward cancer include Glaser, et al., Brit. J. of Cancer, 1999, 80, 756-65; Jing, et al., Leukemia Research, 1999, 23, 43-50; Hoernlein, et al., J. Pharmacol. Exp. Ther., 1999, 288, 613-9; Huang, et al., BioFactors, 2000,13, 225-30; Janssen, et al., Klin. Padiatr., 2000, 212, 189-95; and Winking, et al., J. Neuro-Oncology, 2000, 46, 97-103.

Activity in ileitis and Crohn's disease is reported in Gerhardt, et al., Z. Gastroenterol. 2001, 39, 11-7; Gupta, et al., Planta Med. 2001, 67, 391-5; and Kriegelstein, et al., Int. J. Colorectal, 2001, 16, 88-95.

Other activities reported for the boswellic acids are found in Singh, et al., Phytomedicine, 1996, 3, 87-90; Safayhi, et al., J. Pharmacol. Exp. Ther., 1997, 281, 460-3; Martelli, et al., Int. J. Cosmetic Science, 2000,22,201-6; Syrovets, et al., Mol. Pharmacology, 2000, 58, 71-81; Safayahi, et al., Planta Medica, 2000, 66, 110-3; and Dahmen, et al., Transplantation Proceedings,2001,33,539-41. Wildfener, et al., Arzneim-Forsch DrugRes., 1998, 48, 668-74 describe the acetylation of boswellin and the use of the product in the treatment of experimental autoimmune encephalomyelitis (“EAE”).

Letters patent of interest include: U.S. Pat. Nos. 5,629,351 and 6,174,876; PCT application Nos. WO 97/07796 and WO 00/66111; WO 01/95727, WO 02/085921 A2 and EPA 0 552 657 A1.

### SUMMARY OF THE INVENTION

Compositions having enhanced biological activity are provided by acetylating a purified Boswellia serrata acidic fraction to change the component ratio, thus reducing the content of β-boswellic acid (3-β-hydroxyurs-12-en-23-oic acid; “BA”) and 11-keto-β-boswellic acid (3-β-hydroxyurs-12-en-11-keto-23-oic acid; “KBA”) and enhancing 3-β-acetoxy-β-boswellic acid (3-β-acetoxyurs-12-en-23-oic acid; “ABA”) and 3-β-acetoxy-11-keto-β-boswellic acid (3-β-acetoxyurs-12-en-11-keto-23-oic acid; “AKBA”) content. Further manipulation of this ratio can be achieved by purification and separation techniques, enhancing AKBA content and reducing the ABA content, and/or mild oxidation to convert most of the fraction to the 11-keto derivative, thus enhancing the fraction in AKBA content, the remaining components being primarily other boswellic acids. In certain embodiments, the AKBA is substantially pure. The compositions of AKBA-enriched peracetylated boswellin and AKBA enriched boswellin finds application in a wide variety of treatments. By the term “AKBA-enriched boswellin”, we include a composition having a mixture of boswellic acids that includes a greater percentage being AKBA than is present in a pentacyclic terpene acidic fraction of boswellin.

Compositions comprising boswellic acids can desirably be compatible with lipids, for use in situations in which it is desirable to deliver boswellic acids through the skin, for example. In other situations, it can be desirable to have the boswellic acid in a hydrophilic environment. Salts of boswellic acids can be useful, especially if the solubility of the boswellic acid is sufficiently high to provide an effective concentration of the phytochemical. Solubility of boswellic acid preparations can be increased using a solubility enhancing agent, such as a cyclodextrin.

Preparations comprising boswellic acids can have numerous therapeutic uses including reduction of inflammatory responses and treatment of neoplasias. We unexpectedly found that AKBA enriched peracetylated and AKBA enriched boswellins decreased cancer cell survival in several hormone dependent and independent human prostate and breast cancer cell lines. We found that AKBA enriched boswellin decreased TNF-α production by U937 cells and acts synergistically with other phytochemicals to substantially increase therapeutic effects. In combination with other phytochemicals, AKBA enriched boswellin unexpectedly decreased survival of human cancer cell lines to an extent greater than those produced by the individual phytochemicals alone. Boswellic acids also decreased tumor size and increased survival of animals with transplanted tumors.

Industrial production of boswellic acids includes large scale processing of extracts of Boswellia serrata by further extraction, peracetylation and oxidation to produce substantial quantities of boswellin, peracetylated boswellin and AKBA enriched boswellin for commercial purposes. Improved manufacturing methods permit production of commercial scale aqueous preparations of boswellic acids.

### BRIEF DESCRIPTION OF THE FIGURES

The invention will be described with respect to the particular embodiments thereof. Other objects, features, and advantages of the invention will become apparent with reference to the specification and drawings in which:

FIG. 1 represents chemical structures of four principal boswellic acids comprising a naturally occurring complex, boswellin.

FIG. 2 is a schematic drawing of derivatization and purification procedures of this invention.

FIG. 3 is a schematic drawing of industrial scale extraction of boswellin of this invention.

FIG. 4 is a schematic drawing of industrial scale peracetylation of boswellin of this invention.

FIG. 5 is a schematic drawing of industrial scale oxidation of peracetylated boswellin of this invention.

### DESCRIPTION OF THE SPECIFIC EMBODIMENTS

Biologically active compositions are provided, substantially reduced in BA, ABA and KBA content, enhanced in AKBA content and substantially free of components other than boswellic acids and derivatives found in Boswellia serrata acidic fraction comprised of pentacyclic triterpene acids.

The compositions are directly useful as therapeutic compositions and may be formulated with other components for administration to patients. Of particular interest, is the use of the compositions in the treatment of neoplasia, and inflammation of the musculo-skeletal and other organ systems.

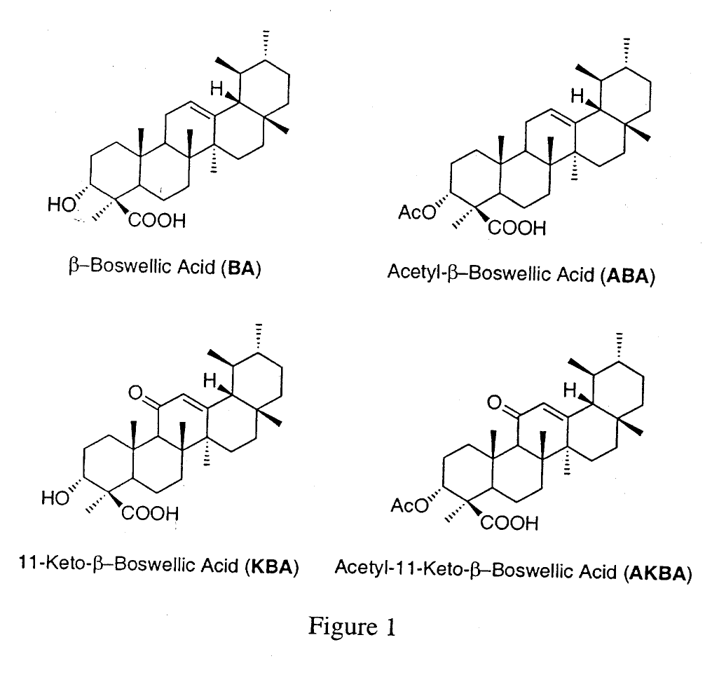

As indicated above, the Boswellia serrata acidic fraction (boswellin) comprised of pentacyclic triterpene acids has four primary components, all derivatives of ursenic acid. FIG. 1 depicts chemical structures of β-boswellic acid (BA), acetyl-β-boswellic acid (ABA), 11-keto-β-boswellic acid (KBA) and acetyl-11-keto-β-boswellic acid (AKBA). Boswellin is available commercially or can be readily isolated by known techniques, each of which may be selected as a matter of convenience. The references indicated above describe methods for isolation of the Boswellia serrata gum and enrichment for the pentacyclic triterpene acid fraction.

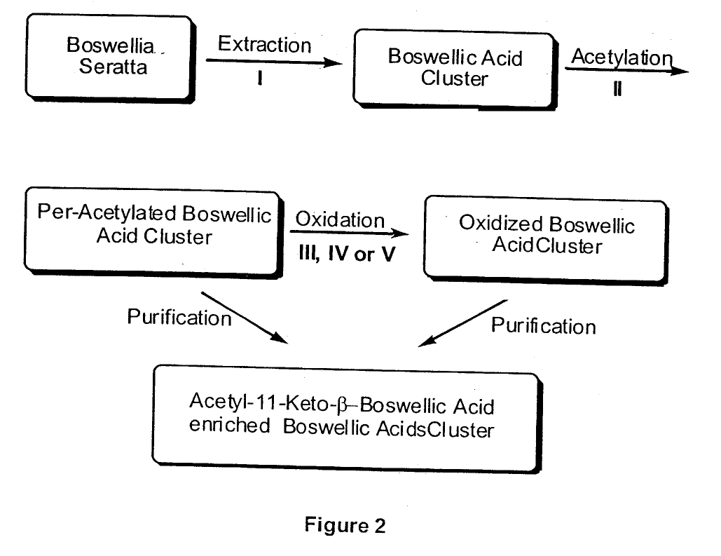

FIG. 2 depicts a diagram showing the procedure for producing AKBA-enriched preparations of boswellin. Generally, an extract will have at least 50% organic acids, preferably at least about 70% organic acids, and will usually have less than about 85% organic acids. In a first stage, the extract (Step 1; Extraction) maybe taken up in a convenient polar solvent, e.g. ethyl acetate and colored materials and adsorbing materials present in the extract removed. Various conventional absorbents may be used, such as activated charcoal, diatomaceous earths, etc. Mild conditions are employed, conveniently 15 to 35° C. The absorbents are then removed, e.g. filtration, the solution extracted with aqueous base, particularly a mild base with a pH below about 9, the aqueous layer isolated and acidified in the presence of a polar organic solvent that is substantially immiscible with water. After drying the organic solvent, the product maybe further cleaned up and dried bypassing through a silica column. The solids are then isolated by evaporation of the solvent. Generally, the resulting product will have about 10 to 30% (wt/wt) of BA, 5 to 20% (wt/wt) of ABA, 5 to 20% (wt/wt) of KBA and 5 to 20% (wt/wt) of AKBA.

The product prepared above is then acetylated under mild conditions (FIG. 2, Acetylation, Step II) to produce peracetylated compositions. The term “peracetylated” means a compound having as many acetyl groups as possible given the chemistry of the acetylation reactions used. Various acetylating agents may be used, such as acetic anhydride, ketene, acetyl halides, mixed acetyl anhydrides, or the like. Methods of acetylating terpenoid alcohols are well known and need not be extensively described here. Conveniently, the product from the first stage is dissolved in a dry polar organic solvent and mixed with the acetylating agent in the presence of a base that serves as an acid neutralizing agent, conveniently a tertiary amine, and at an elevated temperature, usually above about 50° C. and below about 80° C., for a time in the range of about 3 to 9 hours. The reaction is then quenched by cooling, adding a reactive alkanol, e.g. methanol, etc. Volatile components are evaporated and the resulting oil diluted with a polar organic solvent, generally having from about 3-6 carbon atoms, desirably free of reactive functionalities, e.g. hydroxyl. The organic solution is extracted with an aqueous medium, e.g. brine. The organic fraction is dried and evaporated, leaving a crude peracetylated fraction containing an enhanced proportion of AKBA, usually an enhancement of at least about 5% (wt/wt), generally not more enhancement than 15% (wt/wt), an enhanced proportion of ABA, usuallyan enhancement ofat leastabout 15% (wt/wt), generally not more enhancement than 40% (wt/wt), a reduced portion of BA, usually a reduction of at least about 15% (wt/wt), generally a reduction of not more than about 40% (wt/wt), and a reduced portion of KBA, usually reduction of at least about 5% (wt/wt), generally a reduction of not more than about 20% (wt/wt).

Further manipulation of the ratios of β-boswellic acids in crude peracetylated boswellin may be achieved, by but not limited to chromatography, crystallization, dialysis, extraction and other separation techniques. For chromatographic separation one such absorbent is silica (60-200 mesh; Baker Chemical Co.). However, other grades are suitable, including chromatographic grade silica having mesh sizes in the ranges of 35-60, alternatively,60-100,70-230,100-200,130-270,and 200-425. Silica chromatography matrices are commercially available from Baker, Aldrich, Merck and Davisil, for example. The pore size is likewise not crucial with either large or small pore size being suitable. The surface area of the silica is also not critical, with either high surface area or low surface area grades being suitable.

A lightly acidified (about 0.005—about 0.05%) organic solvent is employed to dissolve the fraction, which is the purified on the silica column. The acidifying agent is conveniently a water soluble carboxylic acid, e.g. acetic acid. The product is then eluted from the column with a convenient solvent, usually a mixed solvent of a hydrocarbon and a polar organic solvent. The hydrocarbon solvent is conveniently an aromatic solvent, e.g. benzene, toluene, anisole, xylene, etc. The polar organic solvent in this and other situations is a solvent inert under the conditions it is used, which includes esters, e.g. ethyl acetate, and ethyl butyrate, dimethylsulfoxide, butanol, diethyl ether, methanol, etc., where depending on the conditions and purpose, the solvent may be immiscible with water, slightly soluble with water, or miscible with water. The mixed solvent is used as a gradient in the range of about 4% to about 50% polar organic phase. The product is obtained from the combined fractions, for example, by concentration. The residue is then dissolved in a polar organic solvent, water mixed with the solvent, followed by acidification to a pH below about 5, the resulting isolated precipitate, AKBA enriched peracetylated boswellin, where AKBA will be at least about 20% (wt/wt) and usually less than about 50% (wt/wt), more usually less than about 30% (wt/wt), where ABA will frequently be less than about 20% (wt/wt) usually less than about 10% (wt/wt), more usually less than about 5% (wt/wt) and where both BA and KBA will frequently be less than 10% (wt/wt), usually less than about 5% (wt/wt), more usually less than about 3% (wt/wt).

The crude or AKBA enriched peracetylated boswellin composition is now ready for oxidation. There are a large number ofmild oxidants that can be used and are compatible with the product being physiologically acceptable (FIG. 2, Oxidation, Steps III, IV, V). Peroxides, particularly tert-alkyl hydroperoxides of 4 to 8 carbon atoms, active halides, particularly positive halides, more particularly N-substituted halides such as N-bromosuccinimide, chlorosuccinimide and N-iodosuccinimide, metal oxidants, such as CrO₃/pyridine, Cro₃/pyrazole, pyridinium chromate, sodium chromate, and potassium permanganate find use. Specific oxidants include tert-butyl hydroperoxide, N-bromo succinimide, chromium trioxide and hydrogen peroxide are illustrative. Additional catalysts include Cu(I)Cl, Cu(I)Br, Cu(I)I, Cu(II)Cl₂, Co(II) acetate, and CrO₂ heterogeneous catalysts. For example, Magtrieve™ is a special grade of CrO₂ heterogeneous catalyst available from DuPont. It is characterized as a dense (specific gravity: 4.86 gm/cc) crystalline material and is acicular (typically about 0.3 microns long by about 0.03 microns in diameter). The crystals typically have a specific surface area of about 30 m²/gm. It is a strong ferromagnet and has typical coercivity of about 600 Oersteds and specific magnetization of about 80 emu/gm. These properties permit this catalyst to be removed from the reaction mixture using a magnet. It can be appreciated that numerous such heterogeneous catalysts may be suitable for producing compositions of boswellic acid derivatives of this invention.

The reaction conditions will vary with the particular oxidant and are well established for the individual oxidants. The AKBA enriched preparations can be further purified as described above for AKBA enriched peracetylated boswellin to yield final compositions (FIG. 2, Acetyl-11-Keto-β-Boswellic Acid-enriched boswellin). Specific details may be found in the experimental section. The resulting product will have at least about 50% (wt/wt), preferably at least 70% (wt/wt), more preferably at least about 85% (wt/wt), AKBA. Any remaining components will be at least about 90 wt. % boswellic compounds. AKBA enriched peracetylated boswellin and AKBA enriched boswellin compositions decrease cell survival in both hormone dependent and independent prostate and breast cancer cell lines, LNCaP, PC-3, DU145 and MCF7.

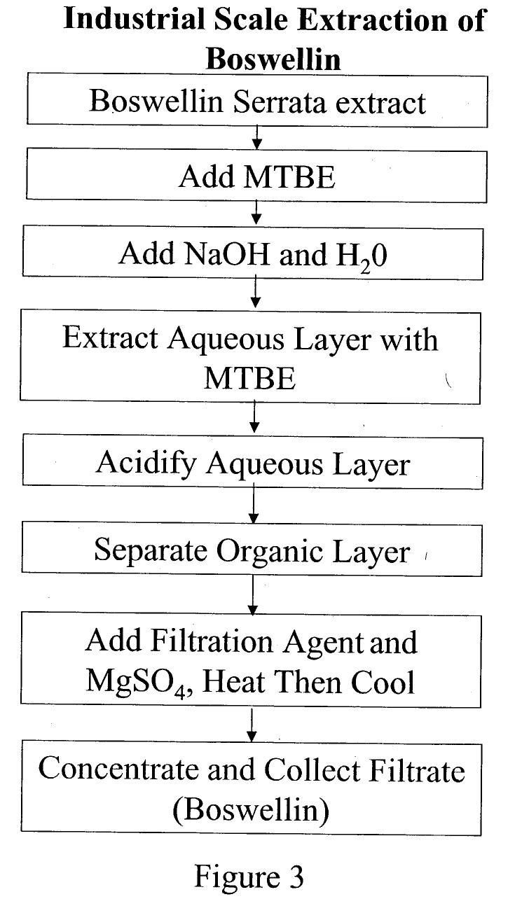

Industrial-scale production of boswellic acids of this invention can be prepared using methods described, in certain embodiments in FIGS. 3, 4 and 5. A crude extract of Boswellia serrata is extracted with an organic solvent, conveniently an ether, and more specifically, a cyclic ether, an alkyl ether, such as diethyl ether, or an alkyl tert-butyl ether such as methyl tert-butyl ether. Although a number of organic solvents can be used, in some situations, it can be desirable to use solvents that have relatively low volatility, compared for example with dimethyl ether. Reducing volatility can reduce hazards from fire.

Peracetylation reactions on an industrial scale can be conveniently carried out using a peracetylating reagent such as acetic anhydride and a pyridine. Although many pyridines may be used, it may be desirable to use 4-(diethylamino)pyridine (DMAP). Subsequent oxidation can be accomplished using any oxidant described herein or using others known in the art. In certain embodiments, it can be desirable to use N-bromosuccinimide. Oxidation can be enhanced by exposure of the reaction mixture to light. In certain embodiments, broad spectrum light can be used. Oxidation reactions can be carried out in glass containers, or, in situations in which large volumes of reactants are to be used, in an enamel-lined container, made, for example of steel. In those situations, exposure to light can be conveniently carried out by irradiating the reactants from above. To stop the oxidation reaction, a reducing agent can be used. Any suitable reducing agent may be used, and in certain embodiments, it can be desirable to use sodium thiosulfate or other thiosulfate salt.

After formation of peracetylated and/or oxidized peracetylated boswellic acids, solutions can be isolated using a filtration agent, such as Filtracel™ and dried using any convenient drying agent, such as MgSO₄ or silica gel.

The ratio of AKBA to the other boswellic acid components in a boswellin mixture appears to alter its effectiveness toward treating proliferative diseases. AKBA enriched boswellin, containing approximately 90% AKBA and AKBA enriched peracetylated material containing a different ratio of boswellic acid components is also active against these cell lines. AKBA enriched boswellin inhibits production of TNF-α in U-937 macrophages, a proinflammatory marker, and when combined with other phytochemicals induces synergistic antineoplastic activity against different types of neoplasias. The combination of AKBA enriched boswellin at 7.5 μg/ml with resveratrol (195 ng/ml), baicalin (12 and 195 ng/ml) or licochalcone A (3.1 μg/ml) inhibited survival of CEM, PC-3 and/or DU145 cancer cells, producing effects surprisingly greater than the sum of the effects of each compound individually. Although the exact mechanisms of synergistic effects are not known, one hypothesis is that the different agents act via different mechanisms but have a final common pathway, namely, regulation of tumor cell proliferation and/or survival. However, other mechanisms of action account for the beneficial effects, and all such mechanisms are considered to be within the scope of this invention.

For treatment, the subject compositions may be formulated in a variety of ways depending on the manner of administration and therapeutic purpose. The composition maybe used as the acid or as a physiologically acceptable salt, such as ammonium, an amine, amino sugar, sodium, potassium, calcium, etc. For a pharmaceutical preparation for oral administration, the product maybe formulated as a tablet or capsule. Various pharmaceutically acceptable additives may be used to obtain particular characteristics for the product. Binding agents include polyvinylpyrrolidone, hydroxypropylmethycellulose, methylcellulose, etc., fillers include lactose, saccharose, mannitol, etc., compaction agents include microcrystalline cellulose and calcium monoacid phosphate, lubricants include stearic acid, polyethylene glycol, magnesium stearate, talc, silicon dioxide, etc., disintegration aiding agents include potato starch, sodium carboxymethylcellulose, etc., wetting agents include sodium lauryl sulfate, etc. The tablets are prepared in accordance with conventional ways.

Other formulations include liquid formulations, such as oil formulations, syrups, elixirs, emulsions, suspensions, etc., or the drug formulation can be provided as a powder for dispersion in an aqueous or other suitable liquid carrier medium. Additives to the liquid medium for suspensions include sorbitol, cellulose derivatives, glucose, gelatin, aluminum stearate, hydrogenated edible fats, etc.; emulsifiers lecithin, gum arabic, sorbitan monooleate, etc; other additives include ethanol, oil of almond, fatty esters, fractionated plant oils. For antioxidants and stabilizers, one may use methyl or propyl paraben, sorbic acid, etc. Other additives include coloring agents, fragrances, sweeteners, etc.

Alternatively, one may formulate the subject compositions as suppositories, inhalants, topical formulations, intramuscular or intravascular injection solutions or suspensions, etc., in accordance with conventional ways, or the like.

Other preparations include lipids. As certain phytochemicals can be extracted using non-polar solvents (e.g., boswellic acids), including such compounds in lipids can be useful for administration across cellular membranes. Additionally, preparations including lipids can be useful for delivery across the skin. In such situations, salves, creams, and ointments can be used.

In other situations, it can be desirable to regulate the absorption of boswellic acids and/or other phytochemicals by the tissue to be treated. Encapsulation of compositions in liposomes or use of slow-release formulations can provide more stable delivery of the desired agents. Many such systems are know to those of skill in the art and need not be described herein further.

The dosage of a boswellic acid composition can generally be in the range of about 0.1—about 200 mg/kg, more usually about 1—about 100 mg/kg, generally per dose in the range of about 0.1 to 200 mg, more usually about 5—about 50 mg per dose, depending upon the purpose of the therapy, the manner administered and the nature of the dose. In many instances, the subject compositions may be used with other compositions in a combination therapy to provide enhanced efficacy. In situations in which slow release of the phytochemical is desired, larger doses can be used, so that over time, the delivery of a desired therapeutic dose in a range described above can be obtained.

The subject compositions have therapeutic effects in a number of indications, such as various neoplasias, systemic or local inflammatory diseases, of organs or organ systems or diseases having a substantial inflammatory component, etc. Various regimens may be employed, giving daily doses of from about 1 to 14 administrations per week. By monitoring the response of the patient, one can determine the effective dosage, although studies in animals have shown that the subject compositions have very little adverse effect, when used appropriately.

The following examples are offered by way of illustration and not by way of limitation.

### EXAMPLES

As described in the examples 1 to 5 below, commercially available Boswellia Serrata gum (Nutriscience lot #BSE-017/9909/B-5) is converted into a peracetylated and AKBA enhanced boswellin in a two or three-step process. Organic extraction of the crude gum, transfer into an alkaline aqueous phase followed by re-acidification and back extraction into an organic phase yields only a slightly purified boswellin. Its acetylation with acetic anhydride (or other acetylating agent) yields a “peracetylated boswellin,” which after purification by silica chromatography produces boswellin usually containing about 20-30% (wt/wt) AKBA where the other β-Boswellic acid components' content has been substantially lowered, and which can be used directly for treatment or enriched further by stoichiometric or catalytic oxidation to yield boswellin containing about 50% (wt/wt) or more AKBA. The content of individual boswellic acids in the modified and unmodified boswellin is given in Table 1.

TABLE 1
Content of Individual Boswellic Acids in Boswellin (mg/g)***

| | BA | ABA | KBA | AKBA |
|---|---|---|---|---|
| Boswellia Serrata* | 165 (38.4) | 101 (23.5) | 69 (16) | 95 (22.1) |
| After Extraction | 201 (39) | 123 (23.8) | 84 (16.3) | 108 (20.9) |
| After Peracetylation** | 11 (3.4) | 28 (8.6) | 13 (4) | 273 (84.0) |
| After Oxidation (tBHP) | 2 (0.5) | 118 (26.9) | n/o (0) | 318 (72.6) |
| After Oxidation (NBS) | n/o (0) | n/o (0) | n/o (0) | 879 (100) |
| After Oxidation (CrO₃) | n/o (0) | n/o (0) | n/o (0) | 736 (100) |

*(Nutriscience lot # BSE-017/9909/B-5)
**After Chromatographic purification.
***Percent values of each of the total β-boswellic acids is given in parentheses
Abbreviations:
BA = β-boswellic acid,
ABA = acetyl-β-boswellic acid,
KBA = 11-keto-β-boswellic acid,
AKBA = acetyl-11-keto-β-boswellic acid,
tBHP = tert-butyl hydroperoxide,
NBS = N-bromo succinimide,
n/o = not observed.

### Example 1

Processing of Boswellia Serrata Gum to Obtain Boswellin (I)

100 g of ground Boswellia Serrata extract (Nutriscience, Lot # BSE-017/9909/B-5, 75% total organic acids) was suspended in 600 ml ethyl acetate, 10 g Norit charcoal, and 10 g Celite. After stirring for 0.5 hour, the mixture was filtered, extracted with 1L Na₂CO₃ (0.5 M), the aqueous layer separated and 4 M HCl in 500 ml butyl acetate added. After drying on MgSO₄, filtration, a silica column (50 gm/60-200 mesh) flushed with 300 ml butyl acetate, produced 81.8 g of product containing 108 mg/g AKBA.

### Example 2

Per-Acetylation of Boswellin (II)

80.00 g of material obtained as above (I) was dissolved in 240 ml ethyl acetate and heated for 5 hours at 70° C. with 50.6 g (0.64 moles) pyridine and 81.7 g (0.80 mol) acetic anhydride, cooled to 40° C. and quenched by 51.3 g (1.6 mol) methanol. Evaporation gave an oil which was diluted with ethyl acetate (500 ml) and extracted with brine (4×300 ml). Upon drying on MgSO₄ and filtration and silica column (40 gm of 60-200 mesh) eluted with 200 ml of a 50/50 ethyl acetate/toluene, evaporation yielded 83.33 g of crude peracetylated boswellin containing 132 mg/g AKBA.

Silica Column Chromatography: A 5.0 g sample of (II) was dissolved in toluene with 0.01% acetic acid (15 mL) and applied to a silica column (35 gm/60-200 mesh), eluted with a gradient of ethyl acetate/toluene/0.01% acetic acid (4%/96% to 50%/50%). Fractions (16×25 mL) were combined and concentrated.

Precipitation: The residue was dissolved in methanol (0.9 mL), added to H₂O (18 mL) and pH adjusted to 3 with 1N HCl. A solid was filtered and dried to yield 1.57 g of AKBA enriched peracetylated boswellin containing 273 mg/g AKBA.

### Example 3

Chromium Oxidation of Per-Acetylated Boswellin (III)

2.93 g (5.8 mmol) of the material obtained (II) (before gradient silica column, 132 mg/g AKBA) was dissolved in 29.3 ml acetic acid, treated with 2.93 g (29.3 mmol) CrO₃ in 52 ml acetic acid, and extracted with 290 ml ethyl acetate, 50 ml water, and 200 ml brine. The ethyl acetate layer was washed (3×) with 100 ml of 50%/50% water/brine, dried on MgSO₄, filtered, and evaporated to yield 3.88 g of material, which was purified by silica column chromatography (as in Example #5) to produce a solid that crystallized from ethyl acetate yielding 1.06 g, containing 736 mg/g AKBA.

### Example 4

Tert-Butyl Hydroperoxide Oxidation of Per-Acetylated Boswellin (IV)

1.04 g (2.1 mmol) (II) (before purification) was dissolved in toluene (12 mL) and treated with 7.5 mL tert-Butyl hydroperoxide (5.6M in decane) and 0.18 g (2.1 mmol) Magtrieve™ at 50° C. After 7 hrs, 13 mL of aqueous sodium sulfite (0.15 g/mL) was added at 0° C. for filtration, followed by extraction with toluene (2×15 mL). After drying over MgSO₄ and concentration, the product dissolved in methanol was precipitated from water yielding 0.87 g containing 318 mg of AKBA.

### Example 5

N-Bromo Succinimide Oxidation of Per-Acetylated Boswellin (V)

25 g of(II),10.0 g (100 mmol) CaCO₃ and 1.20 g (12.5 mmol) methyl cyclohexene were suspended in dioxane (150 mL) and H₂O (3 mL). 22.5 g (126 mmol) N-bromo succinimide was added over 1 hr with an additional 1.73g (18 mmol) methyl-cyclohexene. Upon irradiation with visible light and stirring at RT for 5.5 h, the mixture was filtered, quenched with aqueous sodium sulfite (200 mL, 0.1 g/mL) and concentrated. The residue was suspended in toluene (250 mL), extracted with H₂O (3×200 mL), the organic layer dried over MgSO₄, and concentrated to yield 32 g of product containing 110 mg/g AKBA.

Silica Column Chromatography

30.0 g of the product was dissolved in toluene with 0.01% acetic acid (100 ml) and loaded onto silica column (500 g of 60-200 mesh) which was eluted with 95% toluene/5% ethyl acetate/0.01% acetic acid (5.4 L), 85% toluene/15% ethyl acetate/0.01% acetic acid (3.6 L), and 70% toluene/30% ethyl acetate/0.01% acetic acid (1.8 L). 400 ml fractions were collected, and all fractions containing>5% AKBA (determined by HPLC) were combined and concentrated to yield 7.72 g containing 434 mg/g AKBA.

7.72 g of the combined, concentrated column fractions were dissolved in 8 ml methanol and added drop wise to 1 L of rapidly stirring H₂O. The white solid was filtered off, rinsed with hexane and dried to yield 5.95 g of material containing 448 mg/g AKBA. Crystallization: To 5.8 g ofthe solid in a 50 ml Erlenmeyer flask were added 5.8 ml methanol with 1% H₂O at 55° C. 1.68 g of AKBA enriched boswellin was isolated which contained 879 mg/g AKBA.

### Example 6

Effects of Boswellin Components on Tumor Cells In Vitro

Biological activities of the materials produced as described in Examples 1 to 5 were determined using cultured cells. The IC₅₀s of Boswellin (“BA1”), AKBA enriched peracetylated boswellin (“BA2”) and AKBA enriched boswellin (“BA3”) in various human and murine prostate, leukemia, melanoma, lung and breast cancer cell lines were determined. The IC₅₀s were determined using a standard MTT assay using CEM (lymphoblastic leukemia), Du 145 (prostate cancer), PC-3 (prostate cancer), LNCaP (prostate cancer), Sk-MEL2 (human melanoma), P388D1 (murine leukemia), K562 (human mycloid leukemia), A549 (human lung cancer), MCF-7 (human breast cancer), B16F (murine melanoma) cancer cell lines (Table 2). These results demonstrate that AKBA enriched peracetylated and AKBA enriched boswellin have markedly enhanced antineoplastic activity for specific types of neoplasias. While substantially four to five times more effective than boswellin in the human prostate, DU 145 and PC-3, and hormone dependent breast, MCF7 cancer lines, AKBA enriched peracetylated boswellin has almost ten times more anti-neoplastic activity against LNCaP, hormone-dependent prostate cancer cells.

TABLE 2
Cytotoxic Activities* of Boswellin Components**

| | CEM | DU145 | PC-3 | LNCaP | Sk-MEL2 | P388D1 | K562 | A549 | MCF-7 | B16F |
|---|---|---|---|---|---|---|---|---|---|---|
| BA1 | 205.5 | 225 | 192 | 366.5 | 214 | 146 | 25 | 159 | 221 | 168 |
| BA2 | 53.5 | 48 | 40 | 34.5 | 90 | 33 | 160 | 32 | 54 | 18 |
| BA3 | 59 | 113 | 47 | 37 | 132.5 | 43 | >500 | 33 | 49 | 36 |

*Data obtained using a 3-day MTT assay, and IC₅₀'s are expressed in μg/ml.
**BA1: boswellin; BA2: AKBA enriched peracetylated boswellin; and BA3: AKBA enriched boswellin.

Cell lines in Table 2 are CEM (lymphoblastic leukemia), DU145 (prostate cancer), PC-3 (prostate cancer), LNCaP (prostate cancer), Sk-MEL2 (human melanoma), P388D1 (murine leukemia), K562 (human myeloid leukemia), A549 (human lung cancer), MCF-7 (human breast cancer), B16F (murine melanoma).

### Example 7

Growth Inhibition of LNCaP and Du-145 Cells and Synergy With Genistein

DU145 and LNCaP cells were obtained from ATCC. The cells were grown in Eagle's MEM supplemented with 1 mM sodium pyruvate, 0.1 mM glutamine, 1.5 g/l sodium bicarbonate, and 10% fetal bovine serum. A confluent plate of cells contained approximately 2×10⁶ DU145cells and 10⁶ LNCaP cells.

The test drug dissolved in DMSO at a concentration of 100 mg/ml was added to 6 test plates and to control plates. Two control and two drug plates each were harvested with addition of 1 ml/plate of trypsin-EDTA at 24, 48, and 72 hrs and counted by flow cytometry. The trypsinized plates were incubated for 3 min at 37° C., and 5 ml DMEM was added to each plate. Cells were collected in 15 ml tubes, and 2 ml of each cell suspension was added to 20 ml Isoton and counted.

At a concentration of 100 μg/ml, the AKBA enriched boswellin (approximately 90% AKBA) inhibited the growth of LNCaP and Du-145 cells by 85.5 and 75%, respectively at 24 h, by 89.8 and 85.8%, respectively, at 48 h, and by 94.1 and 87.1%, respectively, at 72 h as compared to controls. Genistein alone in a concentration of 100 μg/ml inhibited LNCaP cell growth by 32.0% at 24 h, 49.4% at 48 h, and by 60.8% at 72 h. When 50 μg/ml of the AKBA enriched boswellin was combined with 50 μg/ml of genistein, the combination inhibited the growth of LNCaP cells by 38.5% at 24 h, 80.4% at 48 h and 90.1% at 72 h as compared to controls, showing synergy between boswellin and genistein.

### Example 8

Inhibition of TNF-α Production After Stimulation of U-937 Macrophages

Human monocyte precursor cells (U937 cells) were grown in RPMI 1640 supplemented with 10% fetal bovine serum and modified with HEPES, sodium bicarbonate and sodium pyruvate, harvested by centrifugation at 200× g, washed twice with phosphate-buffered saline, and resuspended at a density of one million per ml in RPMI 1640 without phenol red and 10% fetal bovine serum which was heat-inactivated and treated with charcoal-coated dextran to remove steroids. Two ml of cell suspension was place in each well of 6-well plates. Cells (except for controls) were activated with phorbol myristate for 24 hrs. Drugs were added at the specified concentration 2 hrs prior to stimulation lipopolysaccharide (LPS) from Samonella Typhimurium. After 24 hrs the supernatant from each well was collected. Cells were removed by centrifugation, and supernatants were analyzed for cytokines by ELISA. Each number is the average of two ELISA determinations.

The AKBA-enriched boswellin (approximately 90% AKBA) from above inhibited the production of TNF-α by 34.8% at 1 μM,51.4% at 10 μM,93.4% at 30 μM, 100% at 60 μM and 100% at 100 μM as compared to untreated controls.

### Example 9

Effects of AKBA Enriched Boswellin (BA3) in Combination With Resveratrol, Baicalin and Licochalcone A on Human Cancer Cell Lines

DU145, PC-3 and CEM cells in culture we studied to determine whether other natural products have synergistic effects with AKBA enriched boswellin. Methods for culturing the cells are described above in Example 6. To different samples of cells, we added AKBA enriched boswellin in concentrations from 0 to 120 μg/ml, resveratrol in concentrations from 0 to 3125 ng/ml, baicalin in concentrations from 0 to 3125 ng/ml and licochalcone A in concentrations from 0 to 800 μg/ml. In analogous concentrations combinations of AKBA enriched boswellin and resveratrol, baicalin and licochalcone A were studied. Cell survival was measured using the MTT assay described above. The results given in Table 3.

From the results of these studies, AKBA enriched boswellin in combination with resveratrol, baicalin and licochalcone A displayed significant synergistic effects on the inhibition of cancer cell survival. The combinations produce an effect greater than the sum of the effects of each compound individually.

TABLE 3
Effects of AKBA Enriched Boswellin (BA3) in Combination With
Resveratrol, Baicalin and Licochalcone A on Human Cancer
Cell Lines.*

| Cancer | % cell | % cell | % cell |
|---|---|---|---|
| Cell | survival with | survival (conc. | survival of the |
| Line | 7.5 μg/ml BA3 | phytochemical) | combination |
| CEM | 69 | 85 (195 ng/ml resveratrol) | 15 |
| CEM | 66 | 85 (195 ng/ml baicalin) | 28 |
| PC-3 | 74 | 98 (3.1 μg/ml licochalcone A) | 26 |
| PC-3 | 92 | 93 (12 ng/ml baicalin) | 50 |
| DU145 | 77 | 89 (195 ng/ml resveratrol) | 42 |
| DU145 | 75 | 78 (195 ng/ml baicalin) | 14 |

*Data from 3-day MTT assay
CEM (lymphoblastic leukemia), DU145 (prostate cancer) and PC-3 (prostate cancer).

### Example 10

Industrial Scale Processing of Boswellia Serrata Gum to Obtain Boswellin (BA1)

FIG. 3 depicts a schematic diagram of an embodiment of this invention for the extraction of commercial quantities of Boswellia serrata. 50 kg of ground Boswellia serrata extract (Laila Impex, Batch Number I 205172, 85% total organic acids) was added to 400 L MTBE, the mixture stirred for 0.5 h, treated with 150 L 3% aqueous NaOH and 500 L water. The aqueous layer was re-extracted with 2×140 L MTBE and the combined aqueous layers stirred with 250 L MTBE and acidified with ˜25 L of 6 M HCl. To the organic layer was added, 2.5 kg Filtracel E™ and MgSO₄, the mixture heated 1 hr at 55° C., cooled, filtered and the filtrate was concentrated (˜180 L). A 200 ml sample of this solution produced 49.44 g (89%) of boswellin (BA1) containing 113 mg/g AKBA and 121 mg/g ABA (9.47% total organic acid content, as COOH; 101.6%, byweight, as average of four major Boswellic Acids, Mᵣ=484.71).

### Example 11

Industrial Scale Peracetylation of Boswellin

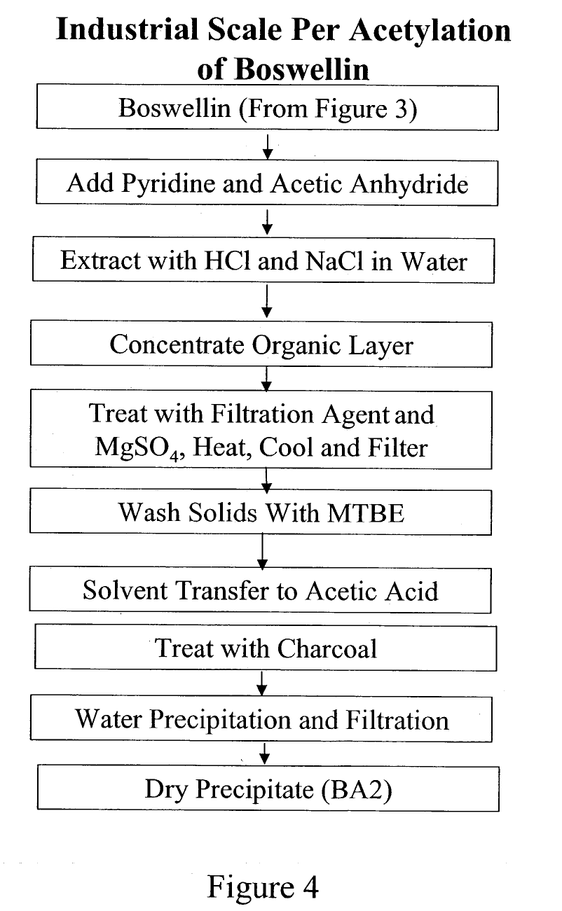

FIG. 4 depicts a schematic diagram of an embodiment of this invention for peracetylation of commercial quantities of Boswellin. 180 L of concentrated Boswellin from above was flushed with N₂ and treated with 2.7 kg (0.022 mol) 4-(dimethylamino)pyridine and 33 kg (0.323 mol) acetic anhydride. The mixture was stirred for 2 h, extracted with 10% HCl (3×60 L) and 20% aqueous NaCl (2×60 L), the organic layer concentrated and treated with 4 kg Filtracel E and MgSO₄. The mixture was heated 1 h at 55° C., cooled, filtered and washed (MTBE). Solvent transfer from MTBE to acetic acid was accomplished by a series of distillations. The final solution was stirred with activated 2 kg charcoal at 70° C., filtered and added slowly to vigorously stirred de-mineralized water (1600 L) at 60° C. The mixture was concentrated under vacuum (˜650 L), 600 L water added, re-concentrated (˜850 L) and stirred 2 h at ambient temperature. The precipitant was collected by filtration and yielded after washing and drying 60° C., 38.5 kg (86.5%) of AKBA enriched enhanced peracetylated boswellin (BA2) containing AKBA (119 mg/g) and ABA (353 mg/g) (10.13% total organic acid content, as COOH; 109.62%, by weight, as average of four major Boswellic Acids). Residual solvents analysis (by GC-head space): max. 140 ppm of MTBE.

### Example 12

Industrial Scale Oxidation of Peracetylated Boswellin

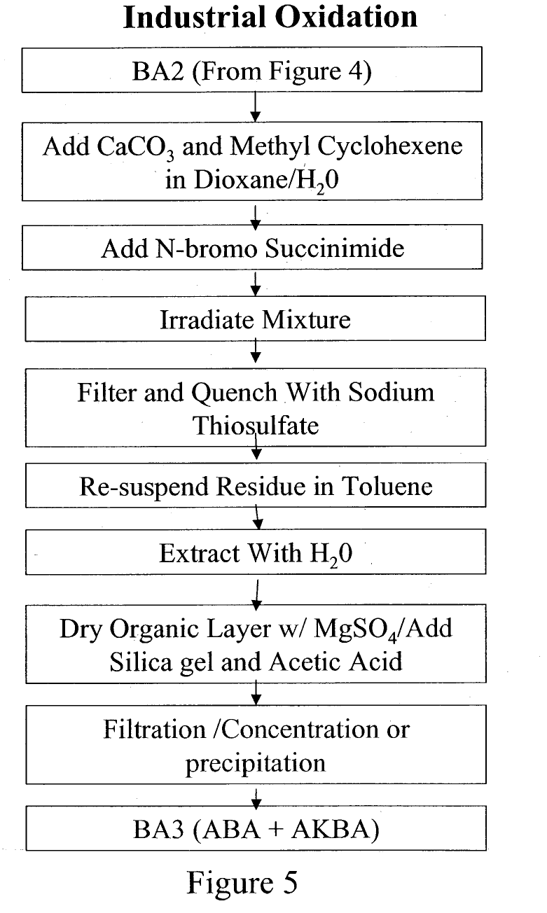

FIG. 5 depicts a schematic diagram of an embodiment of this invention for the oxidation of commercial quantities of peracetylated Boswellin. 660 g of purified BA2, 267 g (2.64 mol) CaCO₃ and 32.0 g (0.33 mol) methyl cyclohexene were suspended in 3.96 L dioxane and H₂O (80 ml). 595 g (3.34 mol) N-bromo succinimide was added over 1 hr with additional methyl cyclohexene (4×15.4 g) every 15 minutes. The mixture was stirred and irradiated with visible light for 5.5 hours, filtered, quenched with aqueous sodium thiosulfate (5.3 L, 0.1 g/ml) and concentrated. The residue was suspended in toluene (6 L), extracted with H₂O (3×6 L), the organic layer dried (MgSO₄) and added to silica gel (80 g, 60 mesh) with ˜1% acetic acid (˜60 ml). The mixture was heated 1 h at 80° C., cooled, filtered through apad of silica gel (300 g) and the silica washed with toluene/1% acetic acid to yield 676 g of AKBA enriched Boswellin (BA3) after concentration or precipitation containing 240 mg/g AKBA and 140 mg/g of ABA. Further AKBA enrichment can be achieved by using silica chromatography as in Example 5 above.

### Example 13

Precipitation of Boswellic Acids Sodium Salts

2 g of BA2 in ethyl acetate was neutralized with NaOH solution in ethanol (1 M) to a pH of 8-9. The precipitation was filtered, washed with ethyl acetate with water (1%) and after drying yielded 1.5 g of the sodium salt of AKBA enriched peracetylated boswellin (BA2/Na).

### Example 14

Effect of Boswellin (BA1), AKBA Enriched Peracetylated Boswellin (BA2) and AKBA Enriched Boswellin (BA3) in C57 BL-6 Mice Bearing Transplanted B16 Melanoma Tumor

B16 Melanoma tumors were transplanted into the upper back of 28 C57 BL-6 mice. After 12 days mice were divided into 4 groups (one control and 3 treatment groups) and the treatment groups administered food laced 0.5% BA1, BA2 or BA3 daily ad libitum for 11 days. Tumor volumes 21 days post implantation and survival times are summarized in Table 4.

TABLE 4
Effect of boswellin (BA1), AKBA enriched peracetylated boswellin (BA2) and AKBA
enriched boswellin (BA3) on melanoma tumors in vivo

| | Control | BA1 | BA2 | BA3 |
|---|---|---|---|---|
| Mean tumor volume (mm³)⁺ | 25590 ± 8832 | 14790 ± 1801 | 12230 ± 1689 | 10000 ± 1135* |
| | n = 2 | n = 7 | n = 3 | n = 6 |
| Mean survival time (days)^{#} | 20.43 ± 1.21 | 23.86 ± 0.34 | 21.71 ± 1.25 | 23.71 ± 0.84 |

⁺Mean tumor volume on day 21 post implantation (mean ± SEM).
*Mean tumor volume statistically different from control (p < 0.05) using a two tailed t-test.
^{#}Mean survival times not statistically different. BA1: boswellin; BA2: AKBA enriched peracetylated boswellin; and BA3: AKBA enriched boswellin.

It is evident from the above results that the therapeutic utility of the natural mixture of boswellic acids can be greatly enhanced using conventional synthetic processes. Because AKBA enriched peracetylated and AKBA enriched boswellin have an altered ratio of enriched components, they are more therapeutically effective than boswellin. The subject invention greatly enhances the utility of the extracts obtained from Boswellia serrata beyond its known anti-inflammatory and anti-neoplastic effects to provide a more diverse, pharmacologically applicable product.

### Example 15

Procedures for Solubilizing Boswellic Acid Mixtures in Aqueous Formulations

(2-hydroxypropyl)-gamma-cyclodextrin (720 mg, 0.456 mmol) and 46.4 mg of peracetylated Boswellin (BA2) were ground in a mortar and pestle. Water (2ml) and 0.16 ml of saturated NaHCO₃was added and the mixture heated in an ultrasonic bath at 60° C. until complete dissolution was achieved (pH ˜7.5 to 8). Material prepared in this fashion was found to be more soluble than BA prepared without cyclodextrin. Thus, this preparation can be more suitably used for either oral or injection administration.

Although the foregoing invention has been described in some detail by way of illustration and example for purposes of clarity of understanding, it will be obvious that certain changes and modifications may be practiced within the scope of the appended claims. All references cited herein are incorporated herein by reference, as if set forth in their entirety.

Industrial Applicability

Compositions are provided comprising boswellin having altered ratios of components by enrichment of active principles (AKBA) relative to other boswellic acids. Methods are provided for peracetylating boswellin as well as enhancing the content of AKBA in boswellin and the use of AKBA enriched peracetylated and AKBA-enriched boswellin preparations in manufacturing pharmacological preparations as drugs or nutritional supplements for prevention or treatment of a variety of neoplastic or inflammatory afflictions.

We claim:

1. A therapeutic composition prepared by the method comprising:
peracetylating a Boswellia serrata pentacyclic terpene acidic fraction extract to provide a peracetylated product; and
optionally oxidizing said peracetylated product with a mild oxidizing agent, to provide a product having a major portion by weight of 3-β-acetoxy-11-keto-β-boswellic acid.

2. A composition according to claim 1, wherein said oxidizing agent is a peroxide.

3. A composition according to claim 1, wherein said peroxide is tert-butyl hydroperoxide.

4. A composition according to claim 1, wherein said oxidizing agent is a metal oxide.

5. A composition according to claim 4, wherein said metal oxide is chromium trioxide.

6. A composition according to claim 4, wherein said oxidizing agent is an active halogen.

7. A composition according to claim 6, wherein said active halogen is an N-substituted halogen.

8. A composition according to claim 7, wherein said N-substituted halogen is N-bromo succinimide.

9. A method of treating a mammalian host for neoplasia, or inflammation, said method comprising:
administering to said mammalian host a therapeutically effective amount of a composition or its physiologically acceptable salts, prepared according to the method comprising:
peracetylating a Boswellia serrata pentacyclic terpene acidic fraction extract to provide a peracetylated product having a ratio of AKBA to the other boswellic acid components in boswellin increased compared to said acidic fraction.

10. A method for treating a mammalian patient for neoplasia, or inflammation, said method comprising:
administering to said mammalian host a therapeutically effective amount of a composition according to claim 1 or its physiologically acceptable salts.

11. A method for enhancing the therapeutic activity of the pentacyclic acidic fraction from Boswellia serrata, said method comprising:
peracetylating said acidic fraction with an acetylating agent to provide a peracetylated product; and optionally
oxidizing said peracetylated product with a mild oxidizing agent,
whereby producing AKBA having enhanced therapeutic activity compared to said pentacyclic acidic fraction.

12. A method according to claim 11, wherein said mild oxidant is a peroxide.

13. A method according to claim 11, wherein said mild oxidant is a metal oxide

14. A method according to claim 11, wherein said mild oxidant is an active halogen.

15. A therapeutic composition, comprising:
a mixture of boswellic acids having AKBA in an amount greater than about 20% of the total boswellic acids by weight;
a solubilizing agent; and
a physiologically compatible carrier.

16. The composition of claim 15, wherein said AKBA is present in an amount greater than about 50% of the total boswellic acids in said mixture.

17. The composition of claim 15, wherein said AKBA is present in an amount greater than about 85% of the total boswellic acids in said mixture.

18. The composition of claim 15, wherein said AKBA is increased by a factor of at least about 90% compared to an unenhanced boswellin.

19. The composition of claim 15, wherein said AKBA is present in an amount of about 100% of the total boswellic acids in said mixture.

20. A tablet comprising the composition of claim 15 and a binder.

21. A capsule comprising the composition of claim 15.

22. A solution comprising the composition of claim 15.

23. A method for treating a mammal suspected of having neoplasia or inflammation, comprising the steps of:
administering to said mammal a therapeutically active amount of a composition of boswellic acids having AKBA or physiologically acceptable salts thereof in an amount greater than about 20% by weight of the total boswellic acids.

24. A method for decreasing production of products of arachidonic acid in a mammal, comprising the step of administering to said mammal a therapeutically effective amount of the composition of claim 15 or one or more of its physiologically acceptable salt.

25. A method for treating inflammation in a mammal, comprising the step of administering to said mammal a therapeutically effective amount of the composition of claim 15 or its physiologically acceptable salts.

26. A method for treating neoplasia in a mammal, comprising the step of administering to said mammal a therapeutically effective amount of the composition of claim 15 or its physiologically acceptable salts.

27. A therapeutic composition, comprising:
a mixture of boswellic acids having BA in an amount less than about 40% of the total boswellic acids by weight and having AKBA in an amount greater than about 20% by weight; and
a physiologically compatible carrier.

28. A therapeutic composition, comprising:
a mixture of boswellic acids having ABA in an amount less than about 25% of the total boswellic acids by weight and having AKBA in an amount greater than about 20% by weight; and
a physiologically compatible carrier.

29. A therapeutic composition, comprising:
a mixture of boswellic acids having KBA in an amount less than about 15% of the total boswellic acids by weight and having AKBA in an amount greater than about 20% by weight; and
a physiologically compatible carrier.

30. A therapeutic composition, comprising:
a mixture of boswellic acids; and
one or more compounds selected from the group consisting of resveratrol, resveratrolosides, genistein, licochalcone A and baicalin;
wherein the combination of said compounds produces an effect greater than the sum of the effects of each compound individually.

31. The composition of claim 30, wherein said effect is anti-neoplastic and/or anti-inflammatory.

32. A method for treating neoplasia in a mammal, comprising the step of administering to said mammal a therapeutically effective amount of the composition of claim 27 or at least one of its physiologically acceptable salts.

33. A method for treating neoplasia in a mammal, comprising the step of administering to said mammal a therapeutically effective amount of the composition of claim 30 or at least one of its physiologically acceptable salts.

34. The method of claim 32, wherein said neoplasia is selected from the group consisting of lymphoblastic leukemia, prostate cancer, lung cancer, melanoma, and breast cancer.

35. A method for inhibiting the production of tumor necrosis factor alpha (TNF-α), comprising exposing to a TNF-α-producing cell, a pharmacologically effective amount of a boswellic acid.

36. The method of claim 35, wherein said boswellic acid is AKBA-enriched boswellin.

37. A method for treating an mammal for a condition characterized by abnormally increased production of TNF-α, comprising administering to said mammal, a therapeutically effective amount of a boswellic acid composition having a concentration of AKBA greater than that present in a pentacyclic acidic fraction of boswellin.

38. A method for treating an animal suffering from neoplasia, comprising administering to said mammal, an amount of AKBA-enriched boswellin sufficient to produce a therapeutic effect in said mammal.

39. The method of claim 38, wherein said therapeutic effect is a decrease in tumor size.

40. The method of claim 38, wherein said therapeutic effect is increase in survival time.

41. The composition of claim 27, wherein said boswellic acids include at least one salt that is solubilized in cyclodextrin.

42. The composition of claim 30, wherein said boswellic acids include at least one salt that is solubilized in cyclodextrin.

43. A method for commercial production of boswellic acids, comprising:
(a) dissolving a crude extract of Boswellia serrata in an organic solvent forming an organic extract;
(b) treating said organic extract with a base and water forming an aqueous layer;
(c) acidifying said aqueous layer forming an acidified aqueous extract;
(d) adding a filtration agent and magnesium sulfate to said acidified aqueous extract; and
(e) filtering said extract produced by step (d).

44. A method for commercial peracetylation of a boswellic acid extract, comprising:
(a) adding a pyridine and an acetic anhydride to a commercial extract of boswellic acids forming a peracetylated boswellic acid fraction in a solvent;
(b) extracting said peracetylated boswellic acid fraction with an inorganic acid and a salt;
(c) adding a filtration agent and magnesium sulfate to said peracetylated boswellic acid fraction;
(d) exchanging said solvent with acetic acid and water, forming a precipitate; and
(d) collecting said precipitate.

45. A method for commercial oxidation of boswellic acids, comprising:
(a) adding calcium carbonate to a peracetylated boswellic acid fraction;
(b) adding methyl cyclohexene to said preparation obtained in step (a);
(c) adding N-bromosuccinimide to the preparation obtained in step (b); and
(d) irradiating said preparation obtained in step (c).

46. A method for forming a sodium salt of peracetylated boswellic acid, comprising:
(a) adding ethyl acetate and ethanol to a preparation of peracetylated boswellic acid; and
(b) adding sodium hydroxide to the preparation obtained in step (a) until a pH of 8 to 9 is reached and a sodium salt of said peracetylated boswellin forms.

47. A method for improving the solubility of boswellic acids, comprising
(a) adding (2-hydroxypropyl)-gamma-cyclodextrin to peracetylated boswellin forming a mixture;
(b) finely dividing the mixture obtained in step (a); and
(c) adding a solution of saturated sodium bicarbonate in water to said finely divided mixture until said mixture dissolves.

48. A composition, comprising:
a mixture of boswellic acids enriched in AKBA compared to an pentacyclic terpene acidic fraction of boswellic acids;
at least one other phytochemical selected from the group consisting of resveratrol, resveratrolosides, genistein, licochalcone A and baicalin; and
a physiologically compatible lipophilic carrier.
